(19) United States
(12) Patent Application Publication
(10) Pub. No.: US 2017/0216273 A1
(43) Pub. Date: Aug. 3, 2017

(54) Nicotine Derivatives and Methods of Use

(71) Applicant: Jory F. Goodman, Los Angeles, CA (US)

(72) Inventor: Jory F. Goodman, Los Angeles, CA (US)

(21) Appl. No.: 15/357,071

(22) PCT Filed: May 11, 2015
(86) PCT No.: PCT/US15/30068

§ 371 Date: Nov. 21, 2016

Related U.S. Application Data
(60) Provisional application No. 62/020,131, filed on Jul. 2, 2014.
(60) Provisional application No. 62/004,504, filed on May 29, 2014.

(51) Int. Cl. A61K 31/465; A61K 31/137; A61K 31/198; A61K 31/519; C07D 401/04; A61K 45/06

(52) U.S. Cl. CPC A61K 31/465; C07D 401/04; A61K 45/06; A61K 31/198; A61K 31/519; A61K 31/137

(57) ABSTRACT

This invention provides a compound selected from the group consisting of L-nicotine(X₁)(X₂) and D-nicotine(X₁) (X₂), or a pharmaceutically acceptable salt thereof, wherein (i) each of X₁ and X₂ is independently a pharmaceutically acceptable anion or a null set; and (ii) X₁ and X₂ cannot both be null sets. This invention also provides related compositions, as well as methods for treating a disorder reasonably believed to be ameliorated by the administration of nicotine.

This application claims priority of U.S. Provisional Application No. 62/004,504, filed May 29, 2014, and U.S. Provisional Application No. 62/020,131, filed Jul. 2, 2014, the contents of both of which are incorporated herein by reference in their entirety.

Throughout this application, various publications are cited. The disclosure of these publications is hereby incorporated by reference into this application to describe more fully the state of the art to which this invention pertains.

### FIELD OF THE INVENTION

The present invention relates to therapeutic nicotine derivatives and uses thereof to treat an array of disorders reasonably believed to be ameliorated by the administration of nicotine.

### BACKGROUND OF THE INVENTION

Nicotinic (N) and muscarinic (M) acetylcholine receptors are distributed throughout the brain and body. Acetylcholine is a biogenic amine involved in neuronal transmission and neuro-modulation. These receptors are involved in myriad activities.

There is broad distribution in the brain, from the ventral tegmentum in the frontal lobes to the basal ganglia, nucleus accumbens, meso-limbic system, hypothalamus, and other regions. Receptors in the brain are N-type.

N and M receptors are distributed throughout the body. They are a major element of the autonomic nervous system, particularly the parasympathetic nervous system. Hence, they are involved in virtually all bodily functions and the maintenance of homeostasis. Commonly known involvement is in the gastrointestinal tract, but there is also intense involvement in cardiac function, blood pressure, vasomotor stability, temperature regulation, and other functions. Vagal nerve stimulation affects not only “somatic” functions, but also central nervous system functions involved in phenomena such as epilepsy, biological mood disorders, and arousal.

Tobacco is the natural source of nicotine. Nicotine, as it is found in tobacco, is a highly addictive molecule. Within seven seconds after inhalation, it activates dopaminergic pleasure centers in the nucleus accumbens via nicotinic acetylcholine receptors. It has effects on the N and M receptors throughout the body.

Morbidity and mortality from tobacco use, and dependence on it, are enormous. It is a very difficult addiction to overcome, and the human and economic costs are extraordinary.

In view of the above, there is a strong and unmet need for derivatives of nicotine that, when administered, permit the safe and therapeutic delivery of nicotine free of the toxic substances associated with it in its natural state.

### SUMMARY OF THE INVENTION

This invention provides a compound selected from the group consisting of L-nicotine(X₁⁻)(X₂⁻) and D-nicotine(X₁⁻)(X₂⁻), or a pharmaceutically acceptable salt thereof, wherein (i) each of X₁⁻ and X₂⁻ is independently a pharmaceutically acceptable anion or a null set; and (ii) X₁⁻ and X₂⁻ cannot both be null sets.

This invention also provides a first composition comprising a first and a second embodiment of the subject compound (i.e., a first and second compound), wherein the first compound has the structure L-nicotine(X₁⁻)(X₂⁻), and the second compound has the structure D-nicotine(X₁⁻)(X₂⁻).

This invention also provides a second composition comprising (i) the subject compound (i.e., a first compound) and (ii) a second compound which, either alone or in conjunction with the first compound, is useful for treating a subject afflicted with a disorder reasonably believed to be ameliorated by the administration of nicotine.

This invention provides three pharmaceutical compositions. The first pharmaceutical composition comprises the subject compound (i.e., nicotine derivative) and a pharmaceutically acceptable carrier. The second pharmaceutical composition comprises the first subject composition (i.e., at least one D-nicotine derivative and at least one L-nicotine derivative) and a pharmaceutically acceptable carrier. The third pharmaceutical composition comprises the second subject composition (i.e., at least one nicotine derivative and at least one second active agent) and a pharmaceutically acceptable carrier.

Finally, this invention provides five therapeutic methods.

The first method is a method for treating a disorder reasonably believed to be ameliorated by the administration of nicotine, comprising administering to a subject afflicted with the disorder a therapeutically effective amount of the subject compound.

The second method is a method for treating a disorder reasonably believed to be ameliorated by the administration of nicotine, comprising administering to a subject afflicted with the disorder a therapeutically effective amount of the first subject composition.

The third method is a method for treating a disorder reasonably believed to be ameliorated by the administration of nicotine, comprising administering to a subject afflicted with the disorder a therapeutically effective amount of the second subject composition.

The fourth method is a method for treating a disorder reasonably believed to be ameliorated by the administration of nicotine, comprising administering to a subject afflicted with the disorder (i) a therapeutically effective amount of the subject compound (i.e., a first compound), and (ii) in conjunction therewith, a therapeutically effective amount of a second compound (i.e., either nicotine or another compound) useful for treating a subject afflicted with a disorder reasonably believed to be ameliorated by the administration of nicotine.

The fifth method is a method for treating a disorder reasonably believed to be ameliorated by the administration of nicotine, comprising administering to a subject afflicted with the disorder a therapeutically effective amount of nicotine.

### DETAILED DESCRIPTION OF THE INVENTION

This invention provides numerous therapeutic nicotine derivatives and therapeutic methods of use. These derivatives can act as fast-acting forms of nicotine useful for safely treating an array of disorders, such as neuropsychiatric and psychological disorders.

This invention permits the safe and controlled delivery of nicotine to nicotinic and muscarinic acetylcholine receptors throughout the central nervous system, autonomic nervous system, and body as a whole. This is accomplished without the concurrent and unwanted delivery of contaminants (such as harmful alkaloids) typically associated with nicotine in its natural state.

### Definitions

In this application, certain terms are used which shall have the meanings set forth as follows.

As used herein, “administer”, with respect to a compound or composition, means to deliver to a subject's body via any known method. Specific modes of administration are discussed below.

As used herein, “nicotine” shall include all enantiomers and racemates of the compound having the following structure:

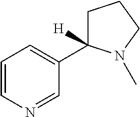

The molecule whose structure is shown above is the L enantiomer (i.e., L-nicotine), and forms the nicotine portion of the derivatives identified herein as L-nicotine(X₁⁻)(X₂⁻). The D enantiomer (i.e., D-nicotine) forms the nicotine portion of the derivatives identified herein as D-nicotine(X₁⁻)(X₂⁻). Naturally occurring nicotine is also referred to in the art as (−)-nicotine and L-(−)-nicotine. Nicotine, as defined herein, is commercially available in its isolated form (e.g., Sigma-Aldrich, St. Louis, Mo.), and is also known as “clean” nicotine, “pure” nicotine and “purified” nicotine (to distinguish it from the forms typically found in nature that are bound to alkaloids and other harmful contaminants). In the preferred embodiment of this invention, and unless stated otherwise herein, the term “nicotine” shall mean any enantiomer or racemate of nicotine in its isolated form having a degree of purity at least as high as that of commercially available medical grade nicotine. In one embodiment, such nicotine purity level is at least 95%, 98%, 99%, 99.5% or 99.9% pure. In another embodiment, such nicotine purity level is 100%. In one embodiment, the commercial medical grade nicotine referred to herein is obtained by known means of removing tars and impurities, and is subjected to purification steps such as steaming or autoclaving followed by repeated sequential micropore filtrations (with or without ultracentrifugation) to remove alkaloids.

As used herein, “nicotine derivative” shall mean nicotine that is non-covalently modified by a chemical moiety such as a halogen ion or hydrogen salt thereof. Nicotine derivatives include, without limitation, the many derivatives discussed below and set forth in Tables 1-3.

As used herein, “pharmaceutically acceptable carrier” shall have the non-limiting embodiments described below.

As used herein, the term “subject” includes, without limitation, a mammal such as a human, a non-human primate, a dog, a cat, a horse, a sheep, a goat, a cow, a rabbit, a pig, a rat and a mouse.

As used herein, a “therapeutically effective amount” of a compound, such as a nicotine derivative, has the following two meanings. If the compound is administered to a subject as the sole active agent, then the therapeutically effective amount is an amount of the compound sufficient to cause a therapeutic result in the subject within a suitable period of time. If the compound is administered to a subject in conjunction with a second active agent (i.e., as part of the same formulation, or in separate formulations administered either concurrently or at different points in time), then the therapeutically effective amount is an amount of the compound sufficient to cause a therapeutic result in the subject within a suitable period of time when administered in conjunction with the second active agent, but not necessarily by itself. Ideally, the therapeutically effective amount of a compound when administered in conjunction with a second active agent is lower than the therapeutically effective amount of the compound when administered as the sole active agent.

As used herein, “treating” a subject afflicted with a disorder shall include, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms. As used herein, “ameliorating” a disorder and “treating” a disorder are equivalent. By way of example, treating a subject includes administering one of the instant compositions to reverse a cluster headache, a migraine headache (via acute rescue), a vasomotor symptom (e.g., Renaud's phenomenon), an attack of acute irritable bowel syndrome, a neuro-cardiogenic problem, or reflux esophagitis.

### Embodiments of the Invention

This invention provides a compound selected from the group consisting of L-nicotine(X₁⁻)(X₂⁻) and D-nicotine(X₁⁻)(X₂⁻), or a pharmaceutically acceptable salt thereof, wherein (i) each of X₁⁻ and X₂⁻ is independently a pharmaceutically acceptable anion or a null set; and (ii) X₁⁻ and X₂⁻ cannot both be null sets. As used herein, “X₁⁻” and “X₂⁻” represent a first anion (or null set) and second anion (or null set), respectively, wherein X₁⁻ represents the anion (or null set) associated with the nitrogen atom in nicotine's five-membered ring, and X₂⁻ represents the anion (or null set) associated with the nitrogen atom in nicotine's six-membered ring.

In a preferred embodiment of the subject compound, each of X₁⁻ and X₂⁻ is independently Br⁻, Cl⁻, F⁻, I⁻, or a null set. Preferably, X₁⁻ and/or X₂⁻ are Br⁻.

When the subject compound is brominated, for example, three permutations exist: (i) X₁⁻ is Br⁻; (ii) X₂⁻ is Br⁻; and (iii) X₁⁻ and X₂⁻ are both Br⁻. As specific embodiments of the subject invention, compounds identified as 1-24 (i.e., the various Br⁻, Cl⁻, F⁻, I⁻ permutations) and racemates identified as 1-12 are set forth in Tables 1-3 below. In the instant compounds, each halogen (e.g., Br⁻) is typically added as a hydrogen salt. Each halogen is attached to the nicotine via an ionic bond. Thus, when the instant compound is ingested, it is hydrolyzed in the stomach to release free nicotine, the free halogen ion and, as applicable, any free cations used to formulate the instant compound (e.g., K⁺, Ca⁺⁺ and Mg⁺⁺).

The D isomer of nicotine is the weaker of the two isomers. It has approximately 25-30% of the L isomer's potency. Side effect incidence and intensity are closely related to potency. Hence, the weaker D isomer has far fewer potential side effects. Racemic nicotine is estimated to have approximately 70-80% of the potency of the L isomer with concomitant potential side effects.

Accordingly, for the nicotine derivatives of this invention, the L isomers are expected to be the most potent and have the most intense side effects. The D isomers are expected to be weaker and have fewer side effects. Racemic formulas are expected to have most of the potency of the L isomers with concomitant potential side effects.

The subject compounds include rapid acting forms useful for acute or rescue therapy, rapid onset and short duration relief of symptoms or disorders, and have a wide variety of delivery options. In a further embodiment, the derivatives are brominated, and are useful for prophylaxis and maintenance.

In this invention, the following derivatives are specifically envisioned: Nicotine-dihydrobromide salt (D, L and R forms); Nicotine-mono-hydrobromide salt (D, L and R forms); and monobromide-Nicotine (D, L and R forms).

Pharmaceutically acceptable salts are well known in the art and include, without limitation, Na⁺, K⁺, Ca⁺⁺, Mg⁺⁺ and various amines (Intl J. Pharm. (1986) 33:201-217).

This invention also provides a first composition comprising a first and a second embodiment of the subject compound (i.e., a first and second compound), wherein the first compound has the structure L-nicotine(X₁⁻)(X₂⁻), and the second compound has the structure D-nicotine(X₁⁻)(X₂⁻).

In a preferred embodiment of the first composition, in each of the first and second compounds, each of X₁⁻ and X₂⁻ is independently Br⁻, Cl⁻, F⁻, I⁻, or a null set. Preferably, X₁⁻ and/or X₂⁻ are Br⁻.

This first composition can contain, for example, a combination of two compounds—an L and a D compound—which are not enantiomers of each other, and thus do not form a racemic mixture together. However, in a preferred embodiment of the first composition, the composition comprises a racemic mixture of the first and second compounds. Various embodiments of racemic forms are set forth in Table 3 below. Non-racemic embodiments of the first composition include, without limitation: (i) D-nicotine(Br₁⁻)+L-nicotine(Br₂⁻); (ii) D-nicotine(Br₁⁻)+L-nicotine(Br₁⁻)(Br₂⁻); (iii) D-nicotine(Br₂⁻)+L-nicotine(Br₁⁻); (iv) D-nicotine(Br₂⁻)+L-nicotine(Br₁⁻)(Br₂⁻); (v) D-nicotine(Br₁⁻)(Br₂⁻)+L-nicotine(Br₁⁻); and (vi) D-nicotine(Br₁⁻)(Br₂⁻)+L-nicotine(Br₂⁻). Additional non-racemic embodiments of the first composition include, without limitation, each of the above embodiments wherein Br⁻ is substituted with Cl⁻, F⁻ or I⁻.

This invention also provides a second composition comprising (i) the subject compound (i.e., a first compound) and (ii) a second compound which, either alone or in conjunction with the first compound, is useful for treating a subject afflicted with a disorder reasonably believed to be ameliorated by the administration of nicotine.

In the preferred embodiment of the second composition, in the first compound, each of X₁⁻ and X₂⁻ is independently Br⁻, Cl⁻, F⁻, I⁻, or a null set. Preferably, X₁⁻ and/or X₂⁻ are Br⁻.

This second composition, in essence, combines one of the instant nicotine derivatives with another, ideally known and FDA-approved, second compound for more effectively treating a suitable disorder. In the preferred embodiment of the second composition, the second compound is selected from the group consisting of (i) an anesthetic (e.g., an anesthetic belonging to the lidocaine, carbocaine or novocaine family); (ii) a neuroleptic (e.g., a phenothiazine (e.g., thorazine, stellazine or navane), a thioxanthene, a butyrophenone (e.g., haldol), an indole, or a dibenzoxazepine); (iii) L-methylfolate; (iv) N-acetylcysteine; (v) a mixture of L-methylfolate and N-acetylcysteine; (vi) Wellbutrin® (bupropion HCl) or Aplenzin® (bupropion HBr); (vii) a dopaminergic agent (e.g., Levodopa, Symmetrel® (amantadine), Permax® (pergolide mesylate), Mirapex® (pramipexole), and Eutonyl® (pargyline)); (viii) an antipsychotic drug (e.g., a dopamine blocker); (ix) an atypical antipsychotic drug (e.g., Risperadal® (risperadone), Zyprexa® (olanzapine), Seroquel® (quetiapine fumarate), and Clozaril® (clozapine)); (x) a major tranquilizer drug; (xi) a minor tranquilizer drug (e.g., a benzodiazepine, a triazolobenzodiazepine, a barbiturate, a carbamate, or a buspirone); (xii) an antidepressant (e.g., a tricyclic compound, a tetracyclic compound, a selective serotonin reuptake inhibitor (“SSRI”), a serotonin and norepinephrine reuptake inhibitor (“SNRI”) (e.g., (Cymbalta® (duloxetine HCl)), or a monoamine oxidase inhibitor (“MAOI”)); (xiii) a mood-stabilizing drug (e.g., lithium, Tegretol® (carbamazepine), Trileptal® (oxcarbezapine), Depakote® (divalproex sodium), Topamax® (topiramate), Zonegran® (zonisamide), Keppra® (levetiracitam)); (xiv) an attention deficit hyperactivity disorder (“ADHD”) drug (e.g., a stimulant, Provigil® (modafinil), Nuvigil® (armodafinil), Ritalin® (methylphenidate HCl)), amphetamine, methamphetamine, or an alpha 2 agonist (e.g., Tenex® (guanfacine HCl) and Catapres® (Clonidine))); (xv) an anticonvulsant drug; (xvi) an anti-seizure drug; (xvii) an anticholinergic agent (e.g., Atropine sulfate); (xviii) an antihistamine (e.g., Benadryl® (diphenhydramine) or Claritin® (loratadine)); (xix) a tryptan; (xx) a proton pump inhibitor; (xxi) an H2 blocker; and (xxii) nicotine.

In a preferred embodiment, the second composition comprises the instant nicotine derivative in a brominated form and aqueous lidocaine, which is administered by drops or insufflation to treat migraine. Certain embodiments of the second composition are set forth in Table 4 below.

This invention provides three pharmaceutical compositions. The first pharmaceutical composition comprises the subject compound (i.e., nicotine derivative) and a pharmaceutically acceptable carrier. The second pharmaceutical composition comprises the first subject composition (i.e., at least one D-nicotine derivative and at least one L-nicotine derivative) and a pharmaceutically acceptable carrier. The third pharmaceutical composition comprises the second subject composition (i.e., at least one nicotine derivative and at least one second active agent) and a pharmaceutically acceptable carrier.

Finally, this invention provides five therapeutic methods.

The first method is a method for treating a disorder reasonably believed to be ameliorated by the administration of nicotine, comprising administering to a subject afflicted with the disorder a therapeutically effective amount of the subject compound.

The second method is a method for treating a disorder reasonably believed to be ameliorated by the administration of nicotine, comprising administering to a subject afflicted with the disorder a therapeutically effective amount of the first subject composition.

The third method is a method for treating a disorder reasonably believed to be ameliorated by the administration of nicotine, comprising administering to a subject afflicted with the disorder a therapeutically effective amount of the second subject composition.

The fourth method is a method for treating a disorder reasonably believed to be ameliorated by the administration of nicotine, comprising administering to a subject afflicted with the disorder (i) a therapeutically effective amount of the subject compound (i.e., a first compound), and (ii) in conjunction therewith, a therapeutically effective amount of a second compound useful for treating a subject afflicted with a disorder reasonably believed to be ameliorated by the administration of nicotine.

The fifth method is a method for treating a disorder reasonably believed to be ameliorated by the administration of nicotine, comprising administering to a subject afflicted with the disorder a therapeutically effective amount of nicotine.

In this invention, every method described herein for employing each of the instant nicotine derivatives (including, without limitation, indication, patient population, dose, and mode of administration) is also envisioned, mutatis mutandis, for nicotine per se.

In the preferred embodiment of each of these five methods, the disorder reasonably believed to be ameliorated by the administration of nicotine is selected from the group consisting of Alzheimer's Disease; ADHD (all subtypes); a migraine headache (all subtypes); a cluster headache; Parkinson's Disease (the full spectrum); a Parkinson spectrum disorder; smoking addiction; tardive dyskinesia (both prophylaxis and treatment); a tardive process; generalized anxiety disorder; panic disorder; obsessive compulsive disorder (OCD); tics; Gilles de la Tourette's Syndrome; P.A.N.D.A.S.; Huntington's Chorea; an adventitious movement disorder (e.g., essential, familial, senile tremor, spastic torticollis, torsion dystonia, traumatic disorder, drug abuse-related disorder, or a post-infectious disorder); schizophrenia; gastro-esophageal reflux disorder (GERD); post-traumatic distress disorder (PTSD); irritable bowel syndrome (IBS); peptic ulcer disease (PUD); epilepsy; a mood disorder; a cardiovascular disorder; a disorder related to vagal nerve stimulation (i.e., a vagal nerve disorder); an autonomic nervous system disorder; and pre-menstrual syndrome.

Preferably, the subject is a human subject. In one embodiment, the human subject is a child, and adolescent, an adult or a geriatric patient. In a preferred embodiment, children and geriatric patients are administered the D isomers of the instant nicotine derivatives, and adolescents and adults are administered the L isomers or racemic forms of the instant nicotine derivatives.

In this invention, administering the instant pharmaceutical compositions can be effected or performed using any of the various methods and delivery systems known to those skilled in the art. The administering can be performed, for example, orally, sublingually, intranasally (by drop or insufflation), intravenously, rectally, via implant, transmucosally, transdermally, intramuscularly, and subcutaneously. In addition, the instant compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant compounds and compositions.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Intranasal administration is a preferred embodiment for the treatment of migraine and cluster headaches in patients who are experiencing nausea and vomiting, are unable to tolerate oral or sublingual administration, and do not prefer rectal delivery.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Transdermal delivery systems include patches, gels, tapes and creams, and can contain excipients such as solubilizers, permeation enhancers (e.g., fatty acids, fatty acid esters, fatty alcohols and amino acids), hydrophilic polymers (e.g., polycarbophil and polyvinylpyrolidone), and adhesives and tackifiers (e.g., polyisobutylenes, silicone-based adhesives, acrylates and polybutene).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Methods of determining therapeutically effective doses for administering the instant compounds and compositions in humans are known in the art. For example, these effective doses can readily be determined mathematically from the results of animal studies.

In one embodiment of the instant invention, the daily dose of nicotine derivative (and where applicable, nicotine) administered to a subject is from 25 μg to 100 mg, and preferably from 250 μg to 45 mg. In another embodiment, the daily dose is selected from one of the following ranges: 25 μg to 50 μg; 50 μg to 100 μg; 100 μg to 250 μg; 250 μg to 500 μg; 500 μg to 1 mg; 1 mg to 5 mg; 5 mg to 10 mg; 10 mg to 25 mg; 25 mg to 45 mg; 25 mg to 50 mg; and 50 mg to 100 mg. In a further embodiment, each of the above dosages is instead administered twice daily, twice weekly, weekly or bi-weekly.

With respect to the “second compound” dosage in the instant compositions and methods, that dosage is preferably the FDA-approved dosage (and dosing regimen where applicable) for that compound.

The instant pharmaceutical compositions can be packaged in the form of pharmaceutical kits or packages in which the daily (or other periodic) dosages are arranged for proper sequential administration. Accordingly, this invention further provides a drug delivery system comprising a pharmaceutical package containing a plurality of dosage units. Each dosage unit is ideally stored in a glass container such as an eye-dropper or ampule, adapted for successive daily, weekly, or other administration, and comprises at least one of the instant pharmaceutical compositions.

This invention will be better understood by reference to the examples which follow, but those skilled in the art will readily appreciate that the specific examples detailed are only illustrative of the invention as described more fully in the claims which follow thereafter.

### Examples

### Example 1: Preparation of Nicotinium Hydrobromide

To a stirred solution of 1.622 g (10 mmol) of nicotine in 50 mL of neat water in an ice bath is added drop-wise 1.131 mL (1.686 g, 10 mmol, 1 eq) of concentrated aqueous hydrobromic acid (ACS reagent, 48%) until the pH is approximately 5.5. The solution is then placed in a lyophilization flask, shell frozen in a bath of liquid nitrogen, and lyophilized to obtain the crude solid nicotinium hydrobromide.

The solid residue is then recrystallized from ethanol/ethyl acetate (EtOH/EtOAc) as follows. Solid nicotinium hydrobromide (10 g, 41.13 mmol) is dissolved in 25 mL of neat EtOH at 40° C. with stirring and then 100 mL of ethyl acetate is added over one minute. The solution is allowed to cool and seeded as necessary to induce the crystallization of nicotinium hydrobromide. The crystals are collected on a Buchner funnel and washed with dry ethyl acetate, and the washes are added to the mother liquor. The combined EtOH/EtOAc solution is reduced to about 20 mL on a rotary evaporator, diluted with dry ethyl acetate (100 mL), and seeded to obtain a second crop of crystals. The combined crystals are dried in vacuo to obtain the solid crystalline nicotinium hydrobromide.

### Example 2: Preparation of Nicotinium Hydrochloride

To a stirred solution of 1.622 g (10 mmol) of nicotine in 50 mL of neat water in an ice bath is added drop-wise 0.821 mL (0.985 g, 10 mmol, 1 eq) of concentrated aqueous hydrochloric acid (ACS reagent, 37%) until the pH is approximately 5.5. The solution is then placed in a lyophilization flask, shell frozen in a bath of liquid nitrogen, and lyophilized to obtain the crude solid nicotinium hydrochloride.

The solid residue is then recrystallized from EtOH/EtOAc as follows. Nicotinium hydrochloride (10 g, 50.3 mmol) is dissolved in 25 mL of neat EtOH at 40° C. with stirring and then 100 mL of ethyl acetate is added over one minute. The solution is allowed to cool and seeded as necessary to induce the crystallization of nicotinium hydrochloride. The crystals are collected on a Buchner funnel and washed with dry ethyl acetate, and the washes are added to the mother liquor. The combined EtOH/EtOAc solution is reduced to about 20 mL on a rotary evaporator, diluted with dry ethyl acetate (100 mL), and seeded to obtain a second crop of crystals. The combined crystals are dried in vacuo to obtain the solid crystalline nicotinium hydrochloride.

### Example 3: Preparation of Nicotinium Dihydrobromide

A stirred solution of 1.622 g (10 mmol) of nicotine in 5 mL of dry EtOH is placed in a round-bottom flask fitted with a drying tube containing calcium chloride beads under argon on an ice bath. To this solution is added drop-wise a solution of 1.699 g (a 5% excess over 20 mmol, 2 eq) of dry hydrogen bromide gas dissolved in 25 mL of dry EtOH. The resulting solution is reduced to about 20 mL on a rotary evaporator, and then 100 mL of ethyl acetate is added over one minute. The solution is allowed to cool and seeded as necessary to induce the crystallization of nicotinium dihydrobromide. The crystals are collected on a Buchner funnel and washed with dry ethyl acetate, and the washes are added to the mother liquor. The combined EtOH/EtOAc solution is reduced to about 20 mL on a rotary evaporator, diluted with dry ethyl acetate (100 mL), and seeded to obtain a second crop of crystals. The combined crystals are dried in vacuo to obtain the crude solid crystalline nicotinium dihydrobromide.

Nicotinium dihydrobromide can be recrystallized from EtOH/EtOAc as follows. Nicotinium dihydrobromide (10 g, 30.86 mmol) is dissolved in 20 mL of neat EtOH at 40° C. with stirring and then 100 mL of ethyl acetate is added over one minute. The solution is allowed to cool and seeded as necessary to induce crystallization. The crystals are collected on a Buchner funnel and washed with dry ethyl acetate, and the washes are added to the mother liquor. The combined EtOH/EtOAc solution is reduced to about 20 mL on a rotary evaporator, diluted with dry ethyl acetate (100 mL), and seeded to obtain a second crop of crystals. The combined crystals are dried in vacuo to obtain the solid crystalline nicotinium dihydrobromide.

### Example 4: Preparation of Nicotinium Dihydrochloride

A stirred solution of 1.622 g (10 mmol) of nicotine in 5 mL of dry EtOH is placed in a round-bottom flask fitted with a drying tube containing calcium chloride beads under argon on an ice bath. To this solution is added drop-wise a solution of 0.766 g (a 5% excess over 20 mmol, 2 eq) of dry hydrogen chloride gas dissolved in 25 mL of dry EtOH. The resulting solution is reduced to about 20 mL on a rotary evaporator, and then 100 mL of ethyl acetate is added over one minute. The solution is allowed to cool and seeded as necessary to induce the crystallization of nicotinium dihydrochloride. The crystals are collected on a Buchner funnel and washed with dry ethyl acetate, and the washes are added to the mother liquor. The combined EtOH/EtOAc solution is reduced to about 20 mL on a rotary evaporator, diluted with dry ethyl acetate (100 mL), and seeded to obtain a second crop of crystals. The combined crystals are dried in vacuo to obtain the solid crystalline nicotinium dihydrochloride.

Nicotinium dihydrochloride can be recrystallized from EtOH/EtOAc as follows. Nicotinium dihydrochloride (10 g, 42.52 mmol) is dissolved in 20 mL of neat EtOH at 40° C. with stirring and then 100 mL of ethyl acetate is added over one minute. The solution is allowed to cool and seeded as necessary to induce crystallization. The crystals are collected on a Buchner funnel and washed with dry ethyl acetate, and the washes are added to the mother liquor. The combined EtOH/EtOAc solution is reduced to about 20 mL on a rotary evaporator, diluted with dry ethyl acetate (100 mL), and seeded to obtain a second crop of crystals. The combined crystals are dried in vacuo to obtain the solid crystalline nicotinium dihydrochloride.

### Example 5: ADHD, all Sub-Types

(a) Children: short-acting therapy, compound 1 or compound 2. Onset sublingually 2-5 minutes. Onset orally 15-30 minutes. Estimated duration of action, 3-5 hours. Areas affected: ventral tegmentum, basal ganglia, limbic system. Probable hypo-thalamic effect. Concurrent autonomic nervous system benefits. Transdermal administration is feasible.

(Pharmacokinetic factors, once described, are not repeated below)

(b) Adolescence: the above can all be considered for adolescence, adults or geriatrics. With the more intense metabolism of adolescents, racemic mixtures should be considered. Thus, for short-acting, compound 1 or racemate 2 can be used. Discussion as above.

(c) Adults: All of the above are applicable to adults, plus the addition of the most potent L isomers. Short-acting racemate 1, compound 2; long-acting compound 1.

### Example 6: Migraine: All Sub-Types

(a) Rescue therapy; children, compound 13 or compound 14. Delivery, sub-lingual, rectal or intra-nasal, with or without lidocaine. Prophylaxis/maintenance same as for ADHD.

(b) Adolescent and adult, rescue therapy compound 1, racemate 1, compound 2, and racemate 2.

Prophylaxis/maintenance is the same as for ADHD.

In migraine, specific receptor activity in the ventral tegmentum, nucleus accumbens, hypothalamus, and vagal nerve are of particular importance.

### Example 7: Cluster Headache

Greater than 90% of cluster headaches are in males. Onset is generally age ten or later. There is an exceptionally strong histaminergic trigger. The most serious complication is suicide. Across the board the most potent isomers are considered. Rescue therapy: compound 3 with either of the racemic mono-bromides as lower potency therapies. Delivery, sub-lingual, rectal or intra-nasal, plus or minus lidocaine. The same compounds, compositions and combinations thereof useful in treating cluster headaches are envisioned for prophylaxis and maintenance. The areas affected are the same as for migraine although the more potent bromination/sedation is an important element in treating the intense stress of cluster headaches, and potent vagal stimulation to diminish autonomic sympathetic activity is also important.

### Example 8: Parkinson's Disease

Therapy for Parkinson's is chronic and of necessity sustained. Therefore, long-acting nicotine derivatives that can be taken on a twice-daily basis are desirable. Primary efficacy is in the basal ganglia although the frontal lobe and miso-limbic activity is critical in arresting, preventing or reversing dementia associated with Parkinson's. Commercially, clinical titrations of the low potency D nicotine derivatives, with or without bromine (from the non-brominated to mono-bromide to di-bromide), would be most desirable, although stronger variations or step therapies from D to R to L are envisioned.

### Example 9: Smoking Cessation

Acute relief provided by sub-lingual mono-bromides. A corollary is a smoking substitute that, in and of itself, would save billions of dollars from the end results of smoking, and can be accomplished with transdermal nicotine derivatives, with or without bromine. Critical activity is in the nucleus accumbens to foster dopaminergic release and in the frontal lobes, and to some degree the temporal lobes, for anxiolysis and to diminish sympathetic activity in the autonomic nervous system (primarily via vagal modulation). Of clinical importance is the desirable avoidance of very rapid release of dopamine, so the sublingual version will have limited use because it will induce too much of a reward sensation.

### Example 10: Tardive Dyskinesia

Tardive dyskinesia results from dopamine blockade in the basal ganglia with concurrent akathisia from effects in the basal ganglia, hypothalamus, frontal and temporal lobes and characteristic numbing or dulling from miso-limbic and nucleus accumbens effects. Desirable treatment can be delivered orally or transdermally on a slow release chronic basis as per Parkinson's' disease. Acute akathisia and possibly acute dystonic reactions can be relieved by sublingual or intra-nasal versions. Activity is in all of the above stated areas. It is postulated that there is some anti-histaminic effect in the hypothalamus as well as peripherally throughout the body. Tardive dyskinesia is an affliction seen most commonly in patients with schizophrenia—of whom there are millions—but also in patients with mood disorders and other individuals who have been exposed to dopamine-blocking agents including common anti-nausea drugs. The comorbidity of tobacco dependence that is extraordinarily high in schizophrenic individuals results in enormous medical complications and huge costs. At the same time, use of the atypical anti-psychotics, which have been proven to be not much better than traditional neuroleptics in the incidence of tardive dyskinesia, also induce metabolic syndrome. Therefore, smoking cessation or substitution in this huge cohort of patients will save billions of dollars annually as the consequences of tobacco use cease. Concurrently, the prevention or relief of tardive dyskinesia will nullify the value of the toxic and very expensive atypical anti-psychotics, and eliminate metabolic syndrome and all of its consequences and costs.

### Example 11: Generalized Anxiety Disorder

At this point, the single best treatment for GAD are the long-acting, low potency benzodiazepines Valium and its congeners, which have a bad reputation and some potential for addiction and abuse. Brominated nicotine derivatives twice daily and titrated to efficacy will be the treatment of choice. Short-acting brominated versions can be used for occasional rescue therapy but should be avoided long-term. The same rationale as described for ADHD can be used for selection of variants across the age span. Primary activity is frontal lobe by diminishing glutamate-transmission and through activation of gabanergic neurons in the temporal lobes and again vigorous activation of the nucleus accumbens.

### Example 12: Panic

Panic attacks must be, but rarely are, distinguished from Panic Disorder. Occasional panic attacks and performance anxiety are best treated with oral or sublingual short-acting brominated nicotine derivatives on an as needed basis. Panic Disorder, on the other hand, is best treated on the front end with rapid suppression by short-acting brominated nicotine derivatives. Areas of activity are the same as for GAD.

### Example 13: Obsessive-Compulsive Disorder (OCD); Tics, Tourette's and Various Presentations of PANDAS

True OCD, not obsessive-compulsive symptoms, is believed to be situated in the cingulate gyrus of the pre-frontal cortex and the anterior temporal lobe. Whether this is idiopathic or PANDAS-related, symptomatology and presentation are the same. If PANDAS-related, attention to the infectious, post-infectious and auto-immune process is absolutely necessary or no effective treatment can be obtained. The same can be said for Tourette's and various tics. Some basal ganglia involvement is probable but unlikely to be the primary driver. The greater the physical component of Tourette's, OCD, Sydenham's Chorea, St. Vitus Dance, and related disorders, the greater the basal ganglia involvement. All of these tend to wax and wane and are exacerbated by stress or anxiety. The transdermally-administered derivatives provide continuous, even release. The degree of bromination and the potency of the isomer or mixture will determine the sedative/anxiolytic affects.

### Example 14: Huntington's Chorea

At this point in time, there is no treatment for this devastating genetic terminal illness. Interest has been expressed by researchers (although without a proposed solution) in the use of nicotine-related compounds for the treatment of Huntington's, if not to cure, then to arrest or slow the progression of the process.

### Example 15: Adventitious Movement Disorders; Tremors; Spastic Torticollis; Torsion Dystonia; Traumatic Post Infectious, Drug Abuse-Related and Idiopathic

This is a group of neurologic disorders that will be treated extensively by physicians by using these nicotine derivatives.

### Example 16: Schizophrenia

It is unclear whether these nicotine derivatives will have a primary therapeutic effect for psychosis per se. However, as stated above, virtually 100% of patients with schizophrenia, schizo-affective disorders and almost all who take dopamine-blocking agents, immediately become heavily tobacco-dependent.

Use of these nicotine derivatives as first line treatment concomitant to neuroleptics will prevent tobacco use, dependence and its sequelae. Also, compositions containing nicotine derivatives and neuroleptics may prevent tardive dyskinesia, and may decrease or resolve tardive processes and akathisia in tardive dyskinesia patients.

### Example 17: Post Traumatic Stress Disorder (PTSD)

PTSD is a specific anxiety disorder. The treatment approach, as with anxiety and panic, would be with an initial combination of short-acting rescue and long-acting prophylactic nicotine derivatives with a transition to maintenance therapy and rare use of rescue therapy. Neurologic receptors are as previously stated.

### Example 18: Peptic Ulcer Disease; Irritable Bowel Syndrome and Gastro Esophageal Reflux Disorder

Each of these is a distinct entity that can be addressed with distinct pharmacotherapies. Physiologically, they are quite similar. Long said to be psychosomatic, they are in fact, neurosomatic. There is substantive involvement of the hypothalamic-pituitary-adrenal axis and marked deregulation of the autonomic nervous system. In actuality, they are part and parcel of many of the things alluded to above. Nonetheless, specific treatments focus on anxiolysis in the central nervous system (frontal lobes), hypothalamic stabilization which directs adrenal modulation and very significantly both vagal nerve activity and muscarinic receptors in the smooth muscle of the gastrointestinal tract. These effects inherently have an impact on acid secretion, gastric emptying and intestinal motility. Both rescue and maintenance therapies can be provided orally and sublingually and even transdermally as warranted.

### Example 19: Additional Disorders

There remain a plethora of illnesses, syndromes, symptoms and disorders. These include, in part, disorders involving the autonomic nervous system and its many aberrations (including such things as Reynaud's Phenomenon) and the central nervous system effects of vagal nerve stimulation. Vagal nerve stimulation is used to treat epilepsies, mood disorders, dysautonomias, cardiac arrhythmias and neuro-cardiogenic syncope. Therefore, a specific subset of the subject nicotine derivatives will be devoted to treating autonomic nervous system and vagal nerve issues.

TABLE 1
Selected Species of L-Nicotine(X₁⁻)(X₂⁻)

| Compound No. | X₁⁻ | X₂⁻ |
|---|---|---|
| 1 | Br⁻ | null |
| 2 | null | Br⁻ |
| 3 | Br⁻ | Br⁻ |
| 4 | Cl⁻ | null |
| 5 | null | Cl⁻ |
| 6 | Cl⁻ | Cl⁻ |
| 7 | F⁻ | null |
| 8 | null | F⁻ |
| 9 | F⁻ | F⁻ |
| 10 | I⁻ | null |
| 11 | null | I⁻ |
| 12 | I⁻ | I⁻ |

TABLE 2
Selected Species of D-Nicotine(X₁⁻)(X₂⁻)

| Compound No. | X₁⁻ | X₂⁻ |
|---|---|---|
| 13 | Br⁻ | null |
| 14 | null | Br⁻ |
| 15 | Br⁻ | Br⁻ |
| 16 | Cl⁻ | null |
| 17 | null | Cl⁻ |
| 18 | Cl⁻ | Cl⁻ |
| 19 | F⁻ | null |
| 20 | null | F⁻ |
| 21 | F⁻ | F⁻ |
| 22 | I⁻ | null |
| 23 | null | I⁻ |
| 24 | I⁻ | I⁻ |

TABLE 3
Selected Species of Racemic Nicotine(X₁⁻)(X₂⁻)

| Racemate No. | X₁⁻ | X₂⁻ |
|---|---|---|
| 1 | Br⁻ | null |
| 2 | null | Br⁻ |
| 3 | Br⁻ | Br⁻ |
| 4 | Cl⁻ | null |
| 5 | null | Cl⁻ |
| 6 | Cl⁻ | Cl⁻ |
| 7 | F⁻ | null |
| 8 | null | F⁻ |
| 9 | F⁻ | F⁻ |
| 10 | I⁻ | null |
| 11 | null | I⁻ |
| 12 | I⁻ | I⁻ |

TABLE 4
Indications And Their Treatment Options

| Indications | Compositions and Remarks |
|---|---|
| Alzheimer's | NHBr alone; NHBr or NHCl + a dopamine |
| Disease | agonist, or a benzodiazepine. N + a dopamine |
| | agonist or a benzodiazepine, or I-methylfolate, |
| | and/or n-acetlycysteine. |
| Dementia | N or any N derivative + I-methylfolate; N or any |
| | N derivative + N-acetylcysteine; or N or any N |
| | derivative + I-methylfolate + N-acetylcysteine. |
| | Deplin (I-methylfolate) is an augmenting agent |
| | for treating major depression. Hence, this |
| | combination can be useful for treating agitated |
| | depression and preempting the need for using |
| | co-therapies with other agents. |
| Attention Deficit | NHBr or NdiHBr alone, or NHCl or NdiHCl |
| Hyperactivity | alone; or any N or N derivative + an |
| Disorder | antidepressant, an alpha 2 agonist, a |
| (ADHD) | benzodiazepine, buspirone, or certain |
| | anticonvulsants. N or any N derivative + |
| | bupropion HCl or HBr. |
| Migraine | NHBr or NdiHBr + local anesthetic (e.g., |
| (including | lidocaine), or an antidepressant (e.g., a tricyclic |
| menstrual | compound, a tetracyclic compound, selective |
| migraine) | serotonin reuptake inhibitor (“SSRI”), a |
| | serotonin and norepinephrine reuptake inhibitor |
| | (“SNRI”) (e.g., (Cymbalta ® (duloxetine HCl)), a |
| | monoamine oxidase inhibitor (“MAOI”), an |
| | anticonvulsant, or a tryptan, or a |
| | benzodiazepine. N or any N derivative + |
| | bupropion HCl or HBr. |
| | Administration modes include, e.g., intranasal |
| | administration. |
| Cluster | NHBr or NdiHBr + local anesthetic (e.g., |
| Headaches | lidocaine), or an antidepressant (e.g., a tricyclic |
| | compound, a tetracyclic compound, selective |
| | serotonin reuptake inhibitor (“SSRI”), a |
| | serotonin and norepinephrine reuptake inhibitor |
| | (“SNRI”) (e.g., (Cymbalta ® (duloxetine HCl)), a |
| | monoamine oxidase inhibitor (“MAOI”), an |
| | anticonvulsant, or a tryptan, or a |
| | benzodiazepine. N or any N derivative + |
| | bupropion HCl or HBr. Administration modes |
| | include, e.g., intranasal administration. |
| Parkinson's | N or any N derivative + a dopamine agonist, or |
| Disease | a benzodiazepine. Any N salt individually. |
| Parkinson | N or any N derivative + a dopamine agonist, a |
| Spectrum | benzodiazepine. Any N salt individually. |
| Disorders | |
| Tardive | N + a major tranquilizer (neuroleptic or |
| Dyskinesia/ | atypical), N + a benzodiazepine. Any N salt |
| Tardive | individually. N or any N derivative + an |
| Processes | antipsychotic drug. Neuroleptics include, for |
| | example, a phenothiazine (e.g., thorazine, |
| | stellazine, or navane), a thioxanthene, a |
| | butyrophenone (e.g., haldol), an indole, and a |
| | dibenzoxazepine. |
| | Prevents Extrapyramidal Symptoms (EPS), |
| | dystonias and tardive processes in treatment- |
| | naïve patients; replaces present therapies in |
| | patients undergoing active treatment without |
| | tardive processes while diminishing side |
| | effects; replaces present therapies in patients |
| | undergoing active treatment with tardive |
| | processes while diminishing or reversing |
| | tardive processes. |
| Tics | Any N salt individually, or N + a |
| | benzodiazepine, an antidepressant, certain |
| | anticonvulsants (gaba-ergics). |
| Tourette's | Any N salt individually, or N + a |
| Syndrome | benzodiazepine, an antidepressant, certain |
| | anticonvulsants (gaba-ergics). |
| Obsessive | Any N salt individually, or N + a |
| Compulsive | benzodiazepine, or an antidepressant. |
| Disorder | |
| Panic Disorder | NHBr or NdiHBr, or N + a benzodiazepine, an |
| | antidepressant (e.g., a tricyclic compound, a |
| | tetracyclic compound, selective serotonin |
| | reuptake inhibitor (“SSRI”), a serotonin and |
| | norepinephrine reuptake inhibitor (“SNRI”) |
| | (e.g., (Cymbalta ® (duloxetine HCl)), a |
| | monoamine oxidase inhibitor ('MAOI”), or |
| | certain anticonvulsants. N or any N derivative + |
| | bupropion HCl or HBr. |
| | The combination of N or N derivative with an |
| | anticonvulsant decreases the required total |
| | dose of medication. Bromine has sedative, |
| | anti-seizure and anti-anxiety effects; reduces |
| | side effects; and may permit the use of lower |
| | doses of anticonvulsant drug. |
| Generalized | NHBr or NdiHBr or N + a minor tranquilizer |
| Anxiety | drug (e.g., a benzodiazepine, a |
| Disorder | triazolobenzodiazepine, a barbiturate, a |
| | carbamate, or buspirone), an antidepressant |
| | (e.g., a tricyclic compound, a tetracyclic |
| | compound, selective serotonin reuptake |
| | inhibitor (“SSRI”), a serotonin and |
| | norepinephrine reuptake inhibitor (“SNRI”) |
| | (e.g., (Cymbalta ® (duloxetine HCl)), a |
| | monoamine oxidase inhibitor (“MAOI”), one of |
| | certain anticonvulsants, or bupropion HCl or |
| | HBr. |
| | The N or N derivative acts synergistically with |
| | the anxiolytic while decreasing the needed |
| | dose of the habituating molecule. For the |
| | combination of nicotine derivative and |
| | buspirone (i.e., Buspar ®), the nicotine |
| | derivative provides early relief while buspirone |
| | (which otherwise has a three-month delay |
| | period before onset of action) exerts its effect |
| | earlier than it would if administered alone. |
| | N or any N derivative + a mood stabilizing drug |
| | (e.g., lithium, Depakote ®, Tegretol ®, |
| | Trileptal ®, Topamax ®, Zolpidem ®, and |
| | Keppra ®). The N and N derivative augments |
| | the effects of mood stabilizers. They decrease |
| | anxiety, decrease total required dose of mood |
| | stabilizing drug, and calm the patient. |
| | N or any N derivative + an ADHD drug (e.g., a |
| | stimulant, Provigil ®, Nuvigil ®, Ritalin ®), an |
| | amphetamine, a methamphetamine, and an |
| | alpha 2 agonist. The N and N derivative |
| | increases the efficacy of the non-nicotine |
| | component. It increases the efficacy of mood |
| | stabilizers and of anti-seizure/anti- |
| | convulsant/anti-epileptic drugs; decreases |
| | anxiety; decreases side effects; and decreases |
| | the total dose of stimulant needed. |
| Post-Traumatic | NHBr or NdiHBr, or N + a benzodiazepine, an |
| Stress Disorder | antidepressant, or certain anticonvulsants. |
| P.A.N.D.A.S. | To be determined by presenting |
| | symptomatology in each case. A majority of |
| | the symptoms of PANDAS are amenable to |
| | treatment. |
| Schizophrenia | N + a neuroleptic (or an atypical, but less |
| | desirable). Any N salt individually added to an |
| | available antipsychotic medication. |
| Epilepsy | N or any N derivative (e.g., a brominated |
| | derivative) + an anticonvulsant, or a |
| | benzodiazepine. |
| | The combination of N or N derivative with an |
| | anticonvulsant decreases the required total |
| | dose of medication. Bromine has sedative, |
| | anti-seizure and anti-anxiety effects; reduces |
| | side effects; and may permit the use of lower |
| | doses of anticonvulsant drug. |
| Major | N + an antidepressant (e.g., a tricyclic |
| Depression | compound, a tetracyclic compound, a selective |
| | serotonin reuptake inhibitor (“SSRI” (least |
| | preferred)), a serotonin and norepinephrine |
| | reuptake inhibitor (“SNRI”) (e.g., (Cymbalta ® |
| | (duloxetine HCl)). N or any N derivative + |
| | bupropion HCl or HBr. |
| Bipolar Disorder | Any N salt individually. N + a mood stabilizer, |
| | an anticonvulsant, a benzodiazepine, or |
| | lithium. |
| Adventitious | N or any N derivative + a dopamine agonist, a |
| Movement | benzodiazepine. Any N salt individually. |
| Disorders (e.g., | |
| Tremor (such | |
| as essential, | |
| familiar and | |
| senile); Torsion | |
| Dystonia; Spastic | |
| Torticollis; and | |
| Spastic | |
| Dysphonia) | |
| Huntington's | Any N salt individually, or N + a |
| Chorea | benzodiazepine. |
| Smoking | Any N salt individually, or N + bupropion HCl, |
| Cessation | or a benzodiazepine. N or any N derivative + |
| | an antidepressant (e.g., a tricyclic compound, a |
| | tetracyclic compound, selective serotonin |
| | reuptake inhibitor (“SSRI”), a serotonin and |
| | norepinephrine reuptake inhibitor (“SNRI”) |
| | (e.g., (Cymbalta ® (duloxetine HCl)), or a |
| | monoamine oxidase inhibitor ('MAOI”). |
| Smoking | Any N salt individually, or N + bupropion HCl, |
| Substitution | or a benzodiazepine. N or any N derivative + |
| | an antidepressant (e.g., a tricyclic compound, a |
| | tetracyclic compound, selective serotonin |
| | reuptake inhibitor (“SSRI”), a serotonin and |
| | norepinephrine reuptake inhibitor (“SNRI”) |
| | (e.g., (Cymbalta ® (duloxetine HCl)), or a |
| | monoamine oxidase inhibitor (“MAOI”). |
| Peptic Ulcer | Any N salt individually, or N + a |
| Disease | benzodiazepine, or certain antidepressants |
| | (doxepin, bupropion, trimipramine), or an H2 |
| | blocker, or a proton pump inhibitor. |
| Irritable Bowel | Any N salt individually, or N + certain |
| Syndrome | antidepressants, or a benzodiazepine. |
| Esophageal | Any N salt individually, or N + a |
| Reflux Disorder | benzodiazepine, or certain antidepressants |
| | (doxepin, bupropion, trimipramine), or an H2 |
| | blocker, or a proton pump inhibitor. |
| Autonomic | Any N salt individually, or N + a |
| Disorders and | benzodiazepine. |
| Dysfunction | |
| (e.g., Reynaud's | |
| Phenomenon) | |
| Vagal Nerve | Any N salt individually, or N + a |
| Issues (e.g., | benzodiazepine or an antidepressant with |
| Epilepsy, and | moderate to significant anticholinergic activity. |
| Treatment- | |
| Resistant | |
| Depression) | |
| Pre-Menstrual | NHBr or NdiHBr + Buproprion HCl or HBr in a |
| Syndrome | proper pulse pattern. N or any N derivative + |
| | an antidepressant (e.g., a tricyclic compound, a |
| | tetracyclic compound, selective serotonin |
| | reuptake inhibitor (“SSRI”), a serotonin and |
| | norepinephrine reuptake inhibitor (“SNRI”) |
| | (e.g., (Cymbalta ® (duloxetine HCl)), or a |
| | monoamine oxidase inhibitor (“MAOI”). |

In this Table, (i) “N”, when not followed by “salt”, represents either nicotine or one of the instant compounds or racemates as defined herein, and (ii) “N salts” represents any of the instant compounds and racemates as defined herein. In this Table, and in this application, it is envisioned that for each embodiment where NHBr or NdiHBr is used in combination with another active agent, NHCl, NdiHCl or any other of the instant compounds may be used in place of the Br form. In this Table, and in this application generally, it is envisioned that for each embodiment where one of the instant compounds or racemates is used alone or in combination with another active agent, nicotine (i.e., not a nicotine derivative as defined herein) may be used in place of the instant compound.

What is claimed is:

1. A compound selected from the group consisting of L-nicotine(X₁⁻)(X₂⁻) and D-nicotine(X₁⁻)(X₂⁻), or a pharmaceutically acceptable salt thereof, wherein (i) each of X₁⁻ and X₂⁻ is independently a pharmaceutically acceptable anion or a null set; and (ii) X₁⁻ and X₂⁻ cannot both be null sets.

2. The compound of claim 1, wherein each of X₁⁻ and X₂⁻ is independently Br⁻, Cl⁻, F⁻, I⁻, or a null set.

3. The compound of claim 2, wherein X₁⁻ and/or X₂⁻ is Br⁻.

4. A composition comprising a first and a second compound of claim 1, wherein the first compound has the structure L-nicotine(X₁⁻)(X₂⁻), and the second compound has the structure D-nicotine(X₁⁻)(X₂⁻).

5. The composition of claim 4, wherein in each of the first and second compounds, each of X₁⁻ and X₂⁻ is independently Br⁻, Cl⁻, F⁻, I⁻, or a null set.

6. The compound of claim 5, wherein X₁⁻ and/or X₂⁻ is Br⁻.

7. The composition of claim 4, wherein the composition comprises a racemic mixture of the first and second compounds.

8. A composition comprising (i) a first compound of claim 1 and (ii) a second compound which, either alone or in conjunction with the first compound, is useful for treating a subject afflicted with a disorder reasonably believed to be ameliorated by the administration of nicotine.

9. The composition of claim 8, wherein the second compound is selected from the group consisting of nicotine; an anesthetic; a neuroleptic; L-methylfolate; N-acetylcysteine; a mixture of L-methylfolate and N-acetylcysteine; bupropion HCl or HBr; a dopaminergic agent; an antipsychotic drug; an atypical antipsychotic drug; a major tranquilizer drug; a minor tranquilizer drug; an antidepressant; a mood-stabilizing drug; an attention deficit hyperactivity disorder (“ADHD”) drug; an anticonvulsant drug; an anti-seizure drug; an anticholinergic agent; an antihistamine; a tryptan; a proton pump inhibitor; and an H2 blocker.

10. The composition of claim 8, wherein in the first compound, each of X₁⁻ and X₂⁻ is independently Br⁻, Cl⁻, F⁻, I⁻, or a null set.

11. The composition of claim 10, wherein in the first compound, X₁⁻ and/or X₂⁻ is Br⁻.

12. A pharmaceutical composition comprising the compound of claim 1 and a pharmaceutically acceptable carrier.

13. A pharmaceutical composition comprising the composition of claim 4 and a pharmaceutically acceptable carrier.

14. A pharmaceutical composition comprising the composition of claim 8 and a pharmaceutically acceptable carrier.

15. A method for treating a disorder reasonably believed to be ameliorated by the administration of nicotine, comprising administering to a subject afflicted with the disorder a therapeutically effective amount of the compound of claim 1.

16. The method of claim 15, wherein the disorder is selected from the group consisting of Alzheimer's Disease; ADHD; a migraine headache; a cluster headache; Parkinson's Disease; a Parkinson spectrum disorder; smoking addiction; tardive dyskinesia; a tardive process; generalized anxiety disorder; panic disorder; obsessive compulsive disorder (OCD); tics; Gilles de la Tourette's Syndrome; P.A.N.D.A.S.; Huntington's Chorea; an adventitious movement disorder; schizophrenia; gastro-esophageal reflux disorder (GERD); post-traumatic distress disorder (PTSD); irritable bowel syndrome (IBS); peptic ulcer disease (PUD); epilepsy; a mood disorder; a cardiovascular disorder; a vagal nerve disorder; pre-menstrual syndrome; and an autonomic nervous system disorder.

17. A method for treating a disorder reasonably believed to be ameliorated by the administration of nicotine, comprising administering to a subject afflicted with the disorder a therapeutically effective amount of the composition of claim 4.

18. The method of claim 17, wherein the disorder is selected from the group consisting of Alzheimer's Disease; ADHD; a migraine headache; a cluster headache; Parkinson's Disease; a Parkinson spectrum disorder; smoking addiction; tardive dyskinesia; a tardive process; generalized anxiety disorder; panic disorder; obsessive compulsive disorder (OCD); tics; Gilles de la Tourette's Syndrome; P.A.N.D.A.S.; Huntington's Chorea; an adventitious movement disorder; schizophrenia; gastro-esophageal reflux disorder (GERD); post-traumatic distress disorder (PTSD); irritable bowel syndrome (IBS); peptic ulcer disease (PUD); epilepsy; a mood disorder; a cardiovascular disorder; a vagal nerve disorder; pre-menstrual syndrome; and an autonomic nervous system disorder.

19. A method for treating a disorder reasonably believed to be ameliorated by the administration of nicotine, comprising administering to a subject afflicted with the disorder a therapeutically effective amount of the composition of claim 8.

20. The method of claim 19, wherein the disorder is selected from the group consisting of Alzheimer's Disease; ADHD; a migraine headache; a cluster headache; Parkinson's Disease; a Parkinson spectrum disorder; smoking addiction; tardive dyskinesia; a tardive process; generalized anxiety disorder; panic disorder; obsessive compulsive disorder (OCD); tics; Gilles de la Tourette's Syndrome; P.A.N.D.A.S.; Huntington's Chorea; an adventitious movement disorder; schizophrenia; gastro-esophageal reflux disorder (GERD); post-traumatic distress disorder (PTSD); irritable bowel syndrome (IBS); peptic ulcer disease (PUD); epilepsy; a mood disorder; a cardiovascular disorder; a vagal nerve disorder; pre-menstrual syndrome; and an autonomic nervous system disorder.

21. A method for treating a disorder reasonably believed to be ameliorated by the administration of nicotine, comprising administering to a subject afflicted with the disorder (i) a therapeutically effective amount of a first compound of claim 1, and (ii) in conjunction therewith, a therapeutically effective amount of a second compound which, either alone or in conjunction with the first compound, is useful for treating a subject afflicted with a disorder reasonably believed to be ameliorated by the administration of nicotine.

22. The method of claim 21, wherein the second compound is selected from the group consisting of nicotine; an anesthetic; a neuroleptic; L-methylfolate; N-acetylcysteine; a mixture of L-methylfolate and N-acetylcysteine; bupropion HCl or HBr; a dopaminergic agent; an antipsychotic drug; an atypical antipsychotic drug; a major tranquilizer drug; a minor tranquilizer drug; an antidepressant; a mood-stabilizing drug; an attention deficit hyperactivity disorder (“ADHD”) drug; an anticonvulsant drug; an anti-seizure drug; an anticholinergic agent; an antihistamine; a tryptan; a proton pump inhibitor; and an H2 blocker.

23. The method of claim 21, wherein the disorder is selected from the group consisting of Alzheimer's Disease; ADHD; a migraine headache; a cluster headache; Parkinson's Disease; a Parkinson spectrum disorder; smoking addiction; tardive dyskinesia; a tardive process; generalized anxiety disorder; panic disorder; obsessive compulsive disorder (OCD); tics; Gilles de la Tourette's Syndrome; P.A.N.D.A.S.; Huntington's Chorea; an adventitious movement disorder; schizophrenia; gastro-esophageal reflux disorder (GERD); post-traumatic distress disorder (PTSD); irritable bowel syndrome (IBS); peptic ulcer disease (PUD); epilepsy; a mood disorder; a cardiovascular disorder; a vagal nerve disorder; pre-menstrual syndrome; and an autonomic nervous system disorder.
